(19) United States
(12) Patent Application Publication
(10) Pub. No.: US 2025/0076284 A1
(43) Pub. Date: Mar. 6, 2025

(54) METHOD FOR EVALUATING BIOLOGICAL ACTIVITY OF BIOMATERIALS

(71) Applicant: Axogen Corporation, Alachua, FL (US)

(72) Inventors: Nesreen ALSMADI, Alachua, FL (US); Kasra TAJDARAN, Alachua, FL (US)

(73) Assignee: Axogen Corporation, Alachua, FL (US)

(21) Appl. No.: 18/808,470

(22) Filed: Aug. 19, 2024

Related U.S. Application Data
(60) Provisional application No. 63/535,685, filed on Aug. 31, 2023.

(51) Int. Cl. G01N 33/50

(52) U.S. Cl. CPC G01N 33/5058; G01N 33/5026

(57) ABSTRACT

A method for conducting an in vitro assay of a biomaterial may include freezing a sample of biomaterial and sectioning the frozen sample of biomaterial into at least one section of biomaterial. The at least one section of biomaterial may have a thickness of about 10 μm to about 50 μm. The method may further include placing the at least one section of biomaterial onto a slide; affixing a neuron or a group of neurons to one end portion of the at least one section of biomaterial to create a test construct; incubating the test construct; and determining an amount of neurite growth from the neuron or the group of neurons along the at least one section of biomaterial.

### CROSS-REFERENCE TO RELATED APPLICATION

This patent application claims the benefit under 35 U.S.C. § 120 to U.S. Provisional Patent Application No. 63/535,685, filed Aug. 31, 2023, the entirety of which is incorporated herein by reference.

### TECHNICAL FIELD

Various embodiments of the present disclosure relate generally to a method for evaluating biological activity of biological materials, and, more particularly, to an in vitro bioassay platform to evaluate potency of biological materials.

### BACKGROUND

To determine the suitability of a tissue graft for surgical repair of damaged or severed tissue, in vitro bioassays may be performed to evaluate potency of a tissue graft, e.g., its ability to facilitate regrowth. Known assays may require more time and cost to conduct. For example, such assays may include affixing a tissue to be regenerated to a three-dimensional, full-thickness graft to form a test construct, culturing the test construct in a medium for a period of time to allow growth of the tissue into the graft, and then sectioning the test construct to attempt to evaluate the extent of the growth into the three-dimensional graft, since growth within the graft cannot be visually assessed without sectioning. The sections may then need to be stained in order to observe the components of the sections. Further, taking sections of the tissue graft may be an imperfect measure, because the exact extent of the tissue growth within the graft may be difficult to assess and may occur between sections. The sections must be sorted through to identify the longest outgrowth of tissue and quantify the growth. Additionally, the neurite growth does not occur in a straight line, making it challenging to accurately track the extent of the growth.

There is a need, however, for a method for evaluating the suitability of a biological material for use in surgical repair that requires less biological material and less time to complete an evaluation. In addition, there is a need for a method in which sections of the biological material are better suited for depositing on a slide used during the analysis, and a method in which analysis does not require sorting through cross-sectional slices to assess the longest outgrowth, and which allows for improved measurement of tissue growth.

The present invention is directed to overcoming one or more of these above-referenced challenges.

### SUMMARY OF THE INVENTION

A method for conducting an in vitro assay of a biomaterial may include freezing a sample of biomaterial and sectioning the frozen sample of biomaterial into at least one section of biomaterial. The at least one section of biomaterial may have a thickness of about 10 μm to about 50 μm. The method may further include placing the at least one section of biomaterial onto a slide; affixing a neuron or a group of neurons to one end portion of the at least one section of biomaterial to create a test construct; incubating the test construct; and determining an amount of neurite growth from the neuron or the group of neurons along the at least one section of biomaterial.

Determining the amount of neurite growth may include measuring at least one longest outgrowing neurite from the neuron or the group of neurons; measuring the at least one longest outgrowing neurite from the neuron or the group of neurons may include measuring a subset of the longest outgrowing neurites and averaging the lengths of the subset of the longest outgrowing neurites; the neuron or each neuron, of the group of neurons, may be a dorsal root ganglia (DRG); the method may further include staining the test construct prior to determining the amount of neurite growth from the neuron or group of neurons; incubating the plurality of sections of biomaterial may include exposing the test construct to carbon dioxide; the method may include using a cryofreezing media when freezing the biomaterial; the slide may have a positive charge on a surface thereof on which the at least one section is placed; the method may include thawing the at least one section of biomaterial and washing the at least one section of biomaterial with a buffer solution prior to affixing the neuron or the group of neurons; the at least one section of biomaterial may include a plurality of sections of biomaterial, and each of the plurality of sections of biomaterial may be placed onto a respective slide; the method may include treating a first subset of the plurality of sections of biomaterial with an agent, and may further comprise determining an effect of the agent on neurite growth by comparing an amount of neurite growth in the treated first subset of sections of biomaterial to an amount of neurite growth in a second subset of untreated sections of the plurality of sections of biomaterial; and the biomaterial may be a nerve graft.

In some aspects, a method for conducting an in vitro assay of a biomaterial may include freezing a sample of biomaterial using a cryofreezing media; sectioning the frozen sample of biomaterial into a plurality of sections of biomaterial, each section having a thickness of about 10 μm to about 50 μm; and placing each section, of the plurality of sections of biomaterial, onto a slide, of a plurality of slides, wherein each slide, of the plurality of slides, has a positive charge on a surface thereof on which a section, of the plurality of sections of biomaterial, is placed. The method may further comprise thawing the plurality of sections of biomaterial; washing the plurality of sections of biomaterial; affixing a neuron or a group of neurons to one end portion of each of the plurality of sections of biomaterial; incubating the plurality of sections of biomaterial, with the neuron or the group of neurons affixed thereto; staining the plurality of incubated sections of biomaterial; and determining, for each of the plurality of incubated sections of biomaterial, an amount of neurite growth from the neuron or the group of neurons along the section of biomaterial.

The method may also include determining, for each of the plurality of incubated sections of biomaterial, the amount of neurite growth may include measuring at least one longest outgrowing neurite from the neuron or the group of neurons; the neuron or each neuron, of the group of neurons, may be a dorsal root ganglia (DRG), and wherein the biomaterial is a nerve graft; the method may include freezing the plurality of sections of biomaterial on the plurality of slides prior to thawing the plurality of sections of biomaterial; the plurality of slides may be frosted or coated with a coating material; the method may include treating a first subset of the plurality of sections of biomaterial with an agent, and further may include determining an effect of the agent on neurite growth by comparing an amount of neurite growth in the treated first subset of sections of biomaterial to an amount of neurite growth in a second subset of untreated sections of the plurality of sections of biomaterial; and incubating the plurality of sections of biomaterial occurs at a temperature of less than 40° C. for about 3 days to about 10 days.

A method for conducting an in vitro assay of nerve tissue may include freezing a sample of nerve tissue using; sectioning the frozen sample of nerve tissue into a plurality of sections of nerve tissue, each section having a thickness of about 10 μm to about 50 μm; placing each section, of the plurality of sections of nerve tissue, onto a positively charged slide, of a plurality of slides; thawing the plurality of sections of nerve tissue; and washing the plurality of sections of nerve tissue. The method may also include affixing a neuron or a group of neurons to one end portion of each of the plurality of sections of nerve tissue; incubating the plurality of sections of nerve tissue, with the neuron or the group of neurons affixed thereto, to allow for neurite growth from the neuron or the group of neurons along the nerve tissue; staining the plurality of incubated sections of nerve tissue; and measuring, on each slide of the plurality of slides, at least one longest outgrowing neurite from the neuron or the group of neurons along the nerve tissue.

### BRIEF DESCRIPTION OF THE FIGURES

The patent or application file contains a least one drawing/photograph executed in color, including FIGS. 9-13, described below. Copies of this patent or patent application publication with color drawing(s)/photograph(s) will be provided by the Office upon request and payment of the necessary fee.

FIG. 1A is a flowchart of a method for conducting an in vitro bioassay, according to the present disclosure.

FIG. 1B is a flowchart of a method for conducting an in vitro bioassay, according to the present disclosure.

FIG. 2 is a schematic illustration of a trimmed sample of biomaterial, according to the present disclosure.

FIG. 3 is a schematic illustration of a plurality of sections of biomaterial, according to the present disclosure.

FIG. 4 is an image of a coated slide.

FIG. 5 is a schematic illustration of a spinal cord having a dorsal root ganglion.

FIG. 6 is a schematic illustration of a dorsal root ganglion affixed to an end of a section of biomaterial on a slide, according to the present disclosure.

FIG. 7 is a schematic illustration of neurite growth from the dorsal root ganglion shown in FIG. 6.

FIG. 8 is a schematic illustration of a covered sample of biomaterial, according to the present disclosure.

FIG. 9 is an image depicting disorganized neurite growth from a dorsal root ganglion.

FIG. 10 is an image depicting organized neurite growth from a dorsal root ganglion.

FIG. 11 is an image of a processed allograft with a dorsal root ganglion affixed thereto, and neurite growth from the dorsal root ganglion along the allograft.

FIG. 12 is an image depicting a processed allograft with a dorsal root ganglion affixed thereto, and neurite growth from the dorsal root ganglion along the allograft.

FIG. 13 is an image depicting a processed allograft with a dorsal root ganglion affixed thereto, and neurite growth from the dorsal root ganglion along the allograft.

### DETAILED DESCRIPTION OF EMBODIMENTS

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ±10% of a specified amount or value unless stated otherwise. As used herein, the terms “comprises,” “comprising,” “includes,” “including,” “has,” “having,” and other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.”

Embodiments of the disclosure are described in relation to a method for conducting an in vitro assay of a biomaterial, and it is contemplated that embodiments of the disclosure may include any suitable type of tissue, including, for example, nerve tissue. Embodiments of the disclosure may include, e.g., grafts of epithelial tissue, connective tissue, vascular tissue, dermal tissue, skeletal tissue, muscle tissue, cardiac tissue, lung tissue, urological tissue, ligament tissue, adipose tissue, connective tissue, or nerve tissue as the biomaterial. Although the examples disclosed herein describe use of “nerves,” “nerve tissue,” “nerve grafts,” or “nerve allografts,” embodiments of the present disclosure may be used to assess any suitable type of tissue. References to a biomaterial may encompass an allograft, a native nerve or native tissue, an engineered tissue graft, a hydrogel with or without one or more bioactives, a synthetic tissue, a natural tissue, or any tissue-based material. If a material is configurable for sectioning, it may be used in the embodiments of the disclosure. In addition, a negatively-charged tissue-based material may be used.

The biomaterials of the present disclosure may also be used with any suitable type of test tissue to assess the potency of the biomaterial, e.g., its ability to promote or otherwise facilitate the growth of tissue to be repaired, including any of the tissue types described above. Although the examples disclosed here describe affixing a neuron or group of neurons, e.g., dorsal root ganglia (DRG), to a biomaterial, the disclosure is not so limited, and any suitable cell or tissue type may be affixed to the biomaterial and assessed for growth.

Embodiments of the disclosure are drawn to a method for conducting an in vitro assay of a biomaterial. As discussed above, it may be desirable to determine the suitability of a tissue graft for repair of damaged or severed tissue prior to in vivo use. Embodiments of the disclosure may be drawn to in vitro assays and methods to evaluate potency or growth of tissue on a tissue graft, and thus to assess the suitability of a tissue graft for use in tissue repair. In some aspects, such assays and methods may be suitable for evaluating a production lot of tissue grafts. In other aspects, assays and methods described herein may be used to evaluate new tissue treatments or the effects of certain experimental substances on tissue growth. In yet other aspects, embodiments of the disclosure may provide a way to easily replicate tissue damage, etc. Additional use cases are set forth in further detail below.

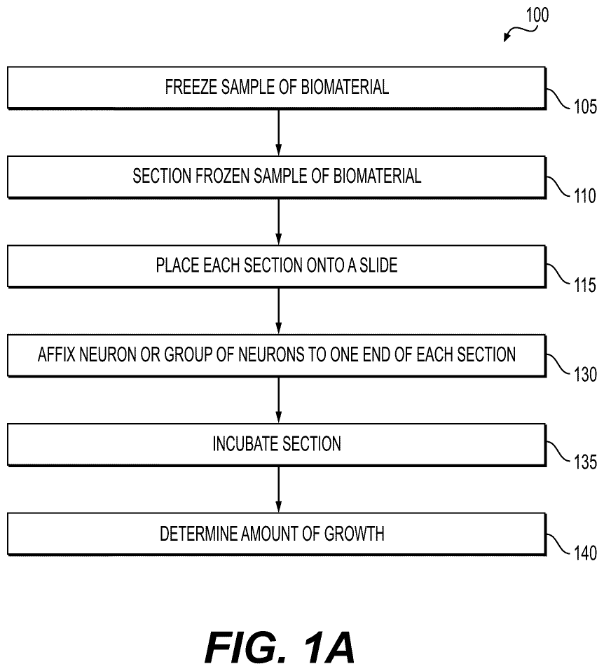

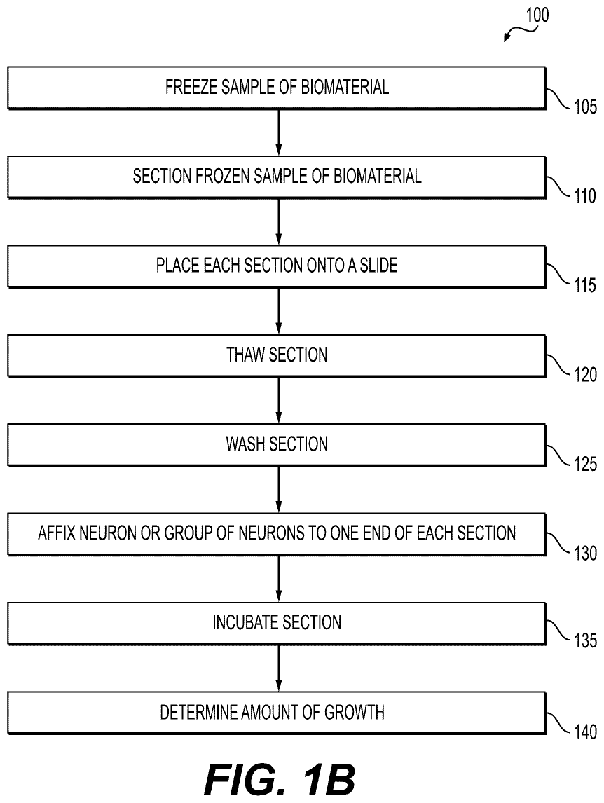

The method of conducting an in vitro assay of a biomaterial, as shown in FIG. 1A, may include the step 105 of freezing a sample of biomaterial. In some aspects, a cryofreezing media may be used during the freezing. As in the example described herein, a suitable biomaterial may be nerve tissue, such as a nerve graft for use in nerve repair. The method may further include step 110 of sectioning the frozen sample of biomaterial into a plurality of sections of biomaterial, each section being of a predetermined thickness, and step 115 of placing each section onto a slide, of a plurality of slides. The method may also include the step 130 of affixing a tissue or cells, such as a neuron or a group of neurons, to one end portion of each of the plurality of sections of biomaterial. The method may further include a step 135 of incubating the plurality of sections of biomaterial with the affixed tissue or cells to allow for growth from the tissue or cells along the biomaterial. The method may also include a step 140 of determining an amount of growth, e.g., neurite growth from the neuron or the group of neurons, across the plurality of sections of biomaterial. Rather than allowing growth of a neuron or group of neurons into a three-dimensional nerve graft (or other tissue) and having to section the nerve graft to assess neurite growth (or the growth of another tissue type), this platform may allow for the analysis of neurite growth on the surface of a thin section of biomaterial. Accordingly, the resulting growth may be observed without sectioning to assess growth.

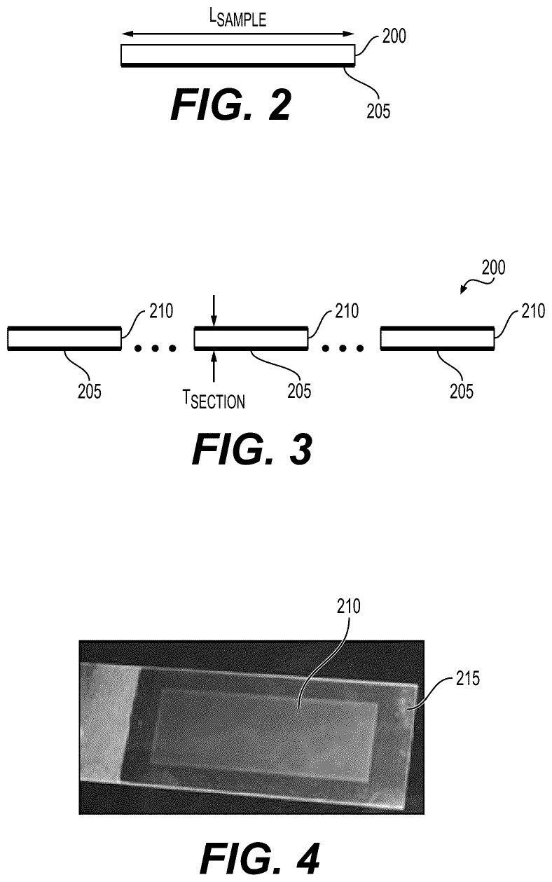

FIG. 1B is a flowchart of a method 100 for conducting an in vitro bioassay, according to the present disclosure. In particular, the method 100 may include a step 105 of freezing a sample 200 of biomaterial, shown in FIG. 2. In some aspects, freezing a sample 200 of biomaterial may include using a cryofreezing media 205. A suitable biomaterial may be any material described above, for example, an engineered tissue graft, a hydrogel with a bioactive in it, synthetic or natural tissue, etc. For example, the biomaterial may be nerve tissue, such as a processed nerve allograft. A cryoprotectant, such as Tissue-TEK® OCT, a water-soluble embedding compound containing glycols and resins that is used for crysectioning (produced by the company Sakura), may be used as the cryofreezing media 205. In some aspects, a sample 200 of biomaterial may be placed on a thin layer of the cryofreezing media 205. FIG. 2 shows the trimmed sample 200 of biomaterial on the layer of cryofreezing media 205. More cryofreezing media 205 may be added to cover the sample 200 of biomaterial. FIG. 8 is a schematic illustration of a covered sample 200 of biomaterial. In some aspects, sufficient amounts of cryofreezing media 205 may be added to the sample 200 of biomaterial to soak or submerge the sample 200 of biomaterial in the cryofreezing media 205. A container, such as a plastic mold, may be used to cover, soak, and/or submerge the sample 200 with the cryofreezing media 205. Step 105 may include flash freezing the biomaterial, for example, using liquid nitrogen.

The method 100 may also include a step 110 of sectioning the frozen sample 200 of biomaterial into at least one section 210 or into a plurality of sections 210 of biomaterial (FIG. 3), each section 210 being of a predetermined section thickness T_{SECTION}. The predetermined section thickness, T_{SECTION}, may be in a range of about 10 μm to about 50 μm. For example, the predetermined section thickness, T_{SECTION}, may be about 10 μm to about 50 μm, about 10 μm to about 40 μm, about 10 μm to about 30 μm, or about 10 μm to about 20 μm. Step 110 of sectioning the sample 200 of biomaterial may include slicing the sample 200 of biomaterial using a slicing implement, such as a knife or a scalpel. In some aspects, about 80 to about 100 sections may be generated, although any suitable number of sections may be generated. The number of sections sample 200 of biomaterial is cut into may depend, e.g., on the size or type of biomaterial 200, or on the use case of the bioassay.

The method may further include a step 115 of placing each section 210, of the plurality of sections 210 of biomaterial, onto a slide 215, of a plurality of slides 215. If only one section is formed in step 110, then the section may be placed on a slide 215. FIG. 4 is an image of a coated slide as one example of such a slide 215. Each slide 215 may have a positive charge on a surface thereof on which a section 210 of biomaterial is placed. The slides 215 may be formed of glass, for example, frosted glass. In addition, the slides 215 may be frosted and/or coated with a coating material (not shown). The coating material may be a material that chemically modifies the properties of the slide 215. As specific examples, 3-Aminopropyl Triethoxysilane (APTS), Laminin-Poly D Lysine, or acid etching may be used as coatings. The coating material may be any material that provides a positive charge, and may be applied to one or more sides of each slide 215. The slides 215 may be any suitable commercially available type of slide. In some embodiments, commercially available slides with a positive charge may be used. Typical samples 200 of biomaterials have a negative charge and are therefore attracted to the positively charged slide 215 surface. Therefore, slides 215 having a positive charge may make the surface of the slide ideal for cell, tissue, and general sample adhesion. That is, cells, tissue, and/or samples of biomaterials described herein having a section thickness of about 10 μm to about 50 μm (as described above) may adhere well to the slides 215 having a positive charge on surfaces thereof. The complimentary charges between the tissue sample and slide may inhibit the tissue sample from floating on the slide.

Once the sections 210 are placed on the slides 215, one or more sections 210 may be thawed for use in a bioassay to test the potency of the tissue samples. Alternatively, after the sections 210 of biomaterial have been placed on slides 215, one or more sections 210 may be placed in a freezer at a relatively low temperature for future use. The temperature of the freezer may be, for example, about −80° C. The slides 215 with the sections 210 of the biomaterial thereon may remain in the freezer at the low temperature for a period of time. For example, the slides 215 may remain in the freezer at the low temperature for up to about 6 months, up to about 1 year, up to about 18 months, or up to about 2 years.

The method 100 may also include a step 120 of thawing one or more of the plurality of sections 210 of biomaterial. Step 120 of thawing the sections 210 of biomaterial may include bringing the sections 210 of biomaterial to a relatively higher temperature, e.g., room temperature, e.g., about 15° C. to about 25° C. In some aspects, the slides 215 with the sections 210 of biomaterial thereon may be placed in a biological hood for thawing.

In addition, the method 100 may include a step 125 of washing the one or more sections 210 of biomaterial. Step 125 of washing one or more sections 210 of biomaterial may be performed to remove extra cryofreezing media 205 from the slides 215. For example, step 125 of washing the sections 210 of biomaterial may be performed until, upon visual inspection, little to no cryofreezing media 205 remains on the slides 215. In some aspects, step 125 may be performed for a predetermined amount of time, for example, less than an hour. Washing may be performed for about 5 minutes to about 30 minutes, e.g., about 10 minutes, about 15 minutes, about 20 minutes, or about 25 minutes. The washing in step 125 may be performed using a solution, such as a buffer solution. As an example, the buffer solution may be a phosphate buffer solution. A buffer solution used in step 125 may have a pH of about 7.4. The solution may be at a temperature in a range of about 15° C. to about 40° C. In one example, the buffer solution may be at room temperature, e.g., about 15° C. to about 25° C.

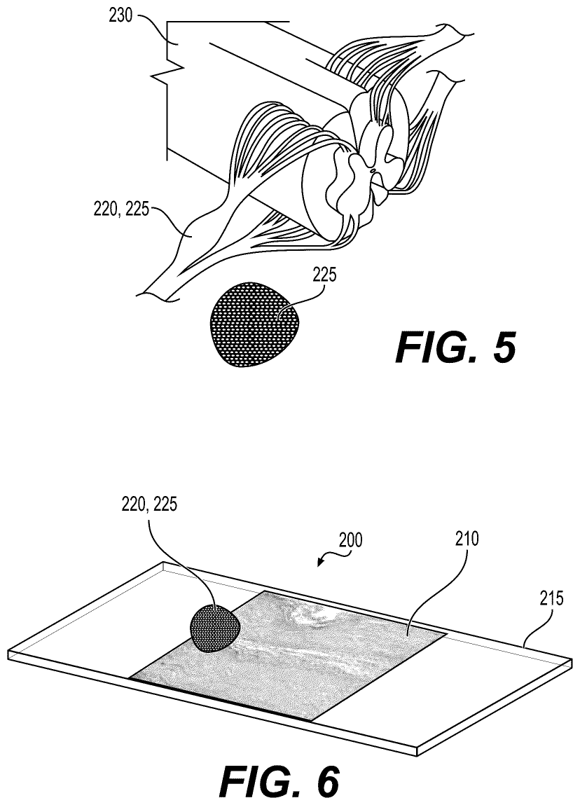

Further, the method 100 may include a step 130 of affixing cells or tissue for re-growing on the biomaterial to test the potency of the biomaterial. The cells or tissue being affixed may be, for example, a neuron or a group of neurons 220. The neuron or group of neurons 220 may be affixed to an end portion of a section 210 of biomaterial. In some aspects, a neuron or group of neurons 220 may be affixed to an end portion of each of the plurality of sections 210 of biomaterial. The neuron or the group of neurons 220 may be a dorsal root ganglion or a group of dorsal root ganglia 225 (collectively (DRG)), respectively. FIG. 5 is a schematic illustration of a spinal cord 230 having dorsal root ganglia 225, and includes a detail view of one dorsal root ganglion 225. The DRG 225, as an example of a neuron 220, may be removed and isolated for use in step 130 of the method 100. FIG. 6 is a schematic illustration of a DRG 225, as an example of a neuron 220, affixed to an end portion of a section 210 of biomaterial on a slide 215. Step 130 of affixing the neuron or the group of neurons 220 may include applying an adhesive (not shown). For example, the adhesive may be collagen or other suitable material. The section 210 of biomaterial with the neuron or group of neurons 220 affixed thereto may be referred to as a test construct 235.

Once the neuron or group of neurons 220 are affixed to the section 210 of biomaterial, the next step 135 may be incubating the test construct 235 for a predetermined amount of time to provide time for the neuron or group of neurons 220 to grow one or more neurites. Incubation step 135 may occur for a predetermined period of time, and at a predetermined temperature. In some aspects, incubation step 135 may occur for several days, for example, for about 3 to about 10 days, e.g., about 3 to about 7 days, about 4 to about 6 days, or about 3, about 4, about 5, about 6, about 7, about 8, about 9, or about 10 days. The amount of incubation time may depend, at least in part, on the type of cell or tissue being grown on the biomaterial, the biomaterial, or the chemicals used in the test construct 235. For example, a biomaterial or test construct 235 including a bioactive may require less incubation time, because growth may occur faster than a biomaterial or test construct 235 not including a bioactive, or a biomaterial or test construct 235 including a growth inhibitor. The predetermined temperature for incubation may be in a range of about 35° C. to about 39° C. In one example, incubation may occur at a temperature of, for example, up to about 40° C., less than 40° C., or about 37° C.

The incubating step may include disposing the plurality of sections 210 of biomaterial in a biological hood where the environment may be controlled or monitored. For example, the pH level of the test construct may be maintained in a range of about 7.3 to about 7.4. Monitoring of the pH level of the test construct may include use of a pH indicator strip and corresponding pH indicator chart. For example, a bright red test result on the pH indicator strip may correspond to and confirm a pH level in the range of 7.3 to 7.4, while a yellow test result indicates that the test construct is acidic, and a purple test result indicates that the test construct is basic. Incubation step 135 may also occur at about 5% CO₂, which may serve as a pH indicator in order to maintain a relatively consistent pH level, or a pH level within a predetermined range.

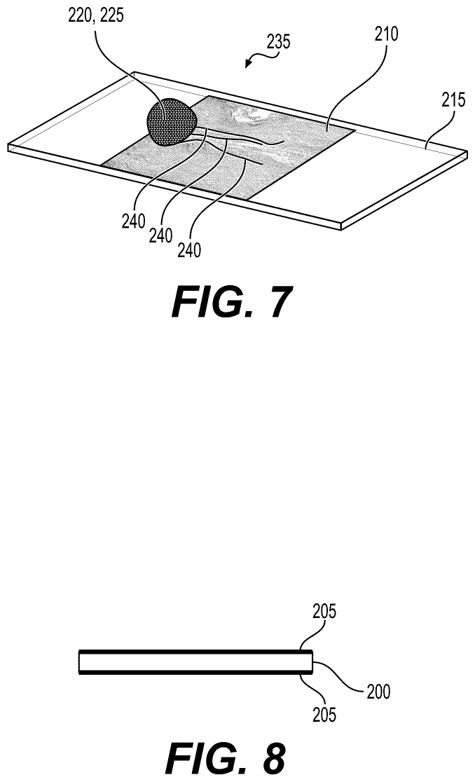

Following incubation, the method 100 may also include a step 140 of determining an amount of neurite growth from the neuron or the group of neurons 220 across, or along the surface of, the sections 210 of biomaterial. Whereas traditional bioassays may use full-thickness tissue grafts or other biomaterials into which regrowth may occur, the thinner sections 210 may allow growth to occur along the surface, or to be visible from the surface, of the sections 210 of biomaterial. As a result, whereas traditional bioassays may require subsequent sectioning or dissection of the test construct 235 to assess growth, this may be avoided with the sections 210. In other words, step 140 may include determining the amount of neurite growth along a given test construct 235. FIG. 7 is a schematic illustration of neurites 240 growing from the DRG 225 shown in FIG. 6, along the section 210 of biomaterial on the slide 215. Step 140 may include measuring at least one longest outgrowing structure (e.g., a neurite 240) from the neuron or the group of neurons 220, or measuring a subset of longest outgrowing structures, or all outgrowing structures. The number of neurites in the subset to be measured may be determined based on one or more factors, as described in more detail below. In some aspects, step 140 may include averaging the length of the measured outgrowing structures in instances in which more than just the longest outgrowing structure is measured.

To obtain measurements, step 140 may include imaging the test constructs 235, scanning the images of the test constructs 235, and performing an analysis on the scanned images using a software program. As an example, the imaging may be diffusion tensor imaging, and the images may be tractography images. As another example, the imaging may be fluorescence microscopy. In some aspects, individual neurites may each be measured, or if the individual neurites are growing too close to each other to easily trace individual neurites, then the longest neurite tip(s) extending the furthest out from the neuron or group of neurons from which they grew may be measured. In some aspects, if scanning images are used, individual neurites may be identified and measured, or the intensity of stain, etc., in the image in certain regions of the test construct may be measured as a stand-in for neurite length.

In one or more embodiments, the method 100 may also include a step of trimming a biomaterial to a predetermined sample length prior to or following step 105 of freezing the sample of biomaterial. FIG. 2 is a schematic illustration of a trimmed sample 200 of biomaterial, having a sample length L_{SAMPLE}. The sample length L_{SAMPLE} may be in a range of about 3 mm to about 25 mm. In one example, the sample length L_{SAMPLE} may be about 10 mm. A suitable length of the sample 200 of biomaterial may depend, at least in part, on the type of biomaterial, the size of the sample 200 prior to trimming, a size or dimension of a slide on which the sample 200 is to be placed, a size or dimension of a slide isolator used to hold the slide with the sample 200 in place, or one or more other aspects of the specific test construct 235 to be prepared.

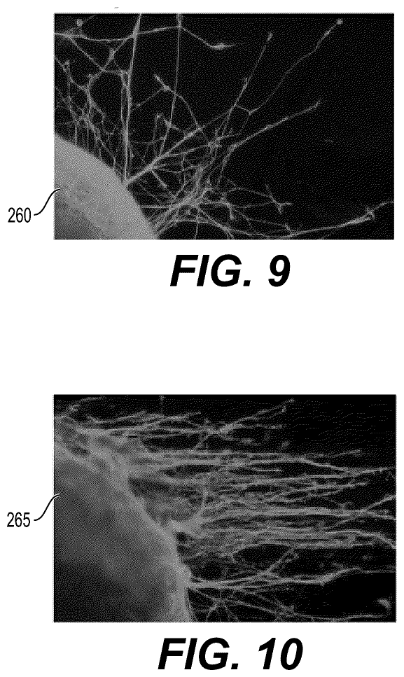

In one or more embodiments, the method 100 may further include performing a histological processing and evaluation of one or more of the plurality of sections 210 of biomaterial, with the neuron or the group of neurons 220 affixed thereto and any resulting neurite outgrowth. The histological processing and evaluation may be performed after incubating the plurality of sections 210 of biomaterial as part of step 140 to determine the amount of outgrowth. The histological processing may include fixing the sections 210, or test constructs 235. Fixing may include disposing the sections 210 (or test constructs 235) in a fixative for a predetermined amount of time. The predetermined amount of time may be, for example, about 30 minutes. The fixative may be, for example, neutral buffered formalin (MBF). Fixing may also include embedding the sections 210 (or test constructs 235) in a fixative. For embedding, the fixative may be, for example, paraffin. The histological processing may also include staining one or more of the plurality of sections 210 of biomaterial with one or more stains. The one or more stains may include Growth Associated Protein 43 (GAP 43) stain, 4′,6-diamidino-2-phenylindole (DAPI) stain, and/or beta III tubulin stain, for example. Staining may aid in visual observation of the neuron or group of neurons and neurite outgrowth, or, if scanning technology is used for step 140, staining may facilitate that process. If other tissue types are used, staining appropriate for those tissue types may be used. Histological evaluation may include observation, measurement, or otherwise assessing the fixed and/or stained sections 210. Examples of stained sections 210 of biomaterial are depicted in FIGS. 9-13, which are discussed below in further detail. Step 140 of determining the amount of neurite growth from the neuron or the group of neurons 220 into the plurality of sections 210 of biomaterial may be performed after one or both of the steps of incubating and performing the histological processing and evaluation. If stain is applied, step 140 of determining the amount of neurite growth may be performed after about 2 days following staining.

As alluded to above, aspects of the disclosure may provide an easier and quicker bioassay to be used as a research platform to compare growth on control, or untreated, slides versus growth on test slides. Instead of using an entire sample 200 of biomaterial to determine growth within the sample 200 and having to assess the growth imprecisely by subsequently sectioning following incubation, each sample 200 of biomaterial can be frozen, sectioned into thin sections 210 having a thickness of about 5 μm to about 50 μm, and a plurality of sections may be generated that are capable of being prepared onto slides. A study can then be run on the plurality of slides more easily, because a greater sample size can be generated from one sample 200 of biomaterial, and since growth occurs along the surface of the thin sections, or is at least visible from the surfaces of the sections 210. As a result, growth can be evaluated quicker and more accurately.

As an example, the method 100 may include a step of treating some or all of the plurality of sections 210 of biomaterial with an agent, e.g., a pharmaceutical agent, a suspected or known growth inhibiter, a suspected or known growth promoter, etc. All sections 210 of biomaterial may be treated, or some sections 210 of biomaterial may be left untreated as controls. Some sections 210 may be treated with different agents, for example, some sections 210 of biomaterial may be treated with a first agent, some sections 210 of biomaterial may be treated with a second agent, and so on, with some or none of the sections 210 of biomaterial being left untreated as controls. In this aspect, the effect on growth of different agents may be compared to one another. Additionally or alternatively, some or all of sections 210 of biomaterial may be altered, e.g., to initiate or simulate injury, e.g., to assess the effect of an agent post-injury. In this way, the embodiments described herein may be used to compare agents, test dosage amounts, compare the effects of different pharmaceuticals, measure inhibitory responses, act as screening tools for bioactives, or be used as a quality control system or to compare differences in donor tissue or among product lots, for example. In other aspects, the effects of different tissue processing methodologies on resulting tissue potency may be compared by assessing any differences in growth between different samples 200 of biomaterial and thus different sections 210 of biomaterial.

In some embodiments, step 140 of determining the amount of neurite growth may include determining an effect of the agent, e.g., pharmaceutical agent, on neurite growth by comparing an amount of neurite growth in one or more treated sections 210 of biomaterial to an amount of neurite growth in one or more untreated sections 210 of biomaterial (i.e., a control), or by comparing an amount of neurite growth in one or more sections 210 of biomaterial treated with a different agent. In such embodiments, the longest neurite in each section 210 of biomaterial may be measured, and the growth of the longest neurite in each treatment section 210 of biomaterial may be compared to the growth of the longest neurite in each control (or second treatment agent) section of biomaterial 210. Alternatively, a predetermined number of the longest neurites in each treatment section 210 of biomaterial may be averaged and compared to the average of the predetermined number of the longest neurites grown in each control (or second treatment agent) section 210 of biomaterial. The predetermined number may be 2 or more (e.g., about 2 to about 50, about 2 to about 30, or about 2 to about 10) of the longest neurites grown in each section 210 of biomaterial. In still other aspects, the number of longest neurites counted in each section 210 of biomaterial may depend on the resulting neurite growth. For example, the number of neurites counted in the section 210 of biomaterial that had the fewest number of neurites that grew may determine the number of the longest neurites will be counted and averaged in each of the other sections 210 of biomaterial. As described above, if neurites cannot be differentiated from one another, then the longest tips extending out from the general neurite growth may be counted.

Although the method 100 is described as including step 105 to step 140, the method 100 may include a subset of these steps. And, as noted above, one or more additional aspects may be included as part of the method 100, including one or more of trimming a biomaterial to a predetermined sample length, placing the biomaterial on the layer of cryofreezing media 205, performing a histological evaluation of the plurality of sections 210 of biomaterial, and treating some or all of the plurality of sections 210 of biomaterial with an agent.

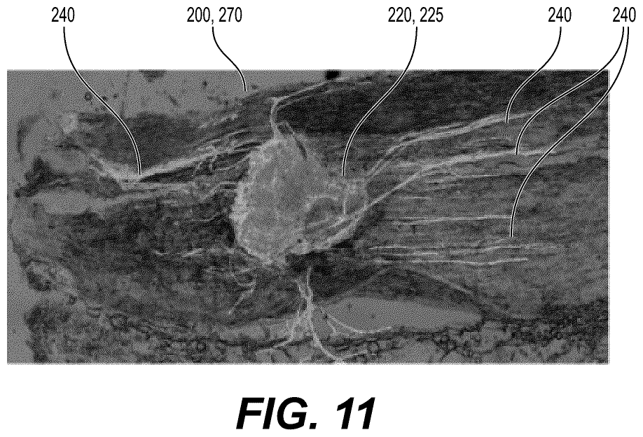

As an example, FIG. 9 is an image depicting a stained section 210 of biomaterial showing disorganized neurite growth, with the neurite growth being shown using GAP-43 stain. DRG 260 was attached to a biomaterial as shown in FIG. 6. The biomaterial on which the neurite growth occurred was not treated with a bioactive, and growth occurred in all directions out from the DRG 260. FIG. 10 is an image depicting a stained section 210 of biomaterial showing organized neurite growth, with the neurite growth being shown using GAP-43 stain. DRG 265 was attached to the biomaterial as shown in FIG. 6. The biomaterial on which the neurite growth occurred was treated with a bioactive, and relatively more organized, directed growth occurred out from the DRG 265.

FIG. 11 is an image of a processed nerve allograft 270, as an example of a sample 200 of biomaterial processed according to the method 100, with a DRG 225, as an example of a neuron 220, affixed thereto. FIG. 11 also shows neurite growth from the DRG 225 into the allograft 270. The DRG 225 and the neurites 240 are emphasized using GAP 43 stain and DAPI stain. In particular, the green portions of the image of FIG. 11 depict neurite growth, or neural outgrowth and regeneration, as emphasized with GAP 43 stain, and the blue portions of the image of FIG. 11 depict nuclear DNA of the DRG 225 and the neurites 240.

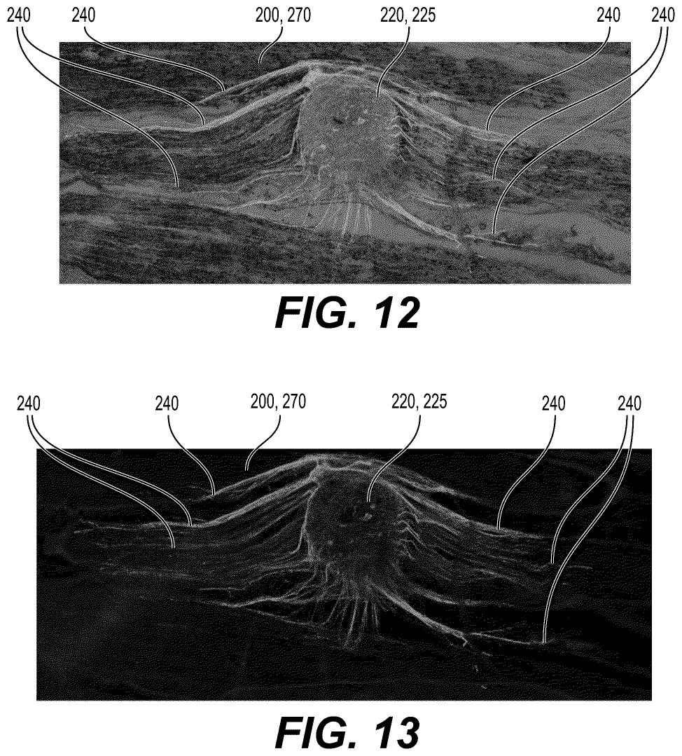

FIG. 12 is another image depicting processed allograft 270, showing neurite growth from DRG 225 into the allograft 270, emphasized using GAP 43 stain and DAPI stain. In particular, the green portions of the image of FIG. 12 depict neurite growth, or neural outgrowth and regeneration, as emphasized with GAP 43 stain, and the blue portions of the image of FIG. 12 depict nuclear DNA of the DRG 225 and the neurites 240. The alignment of neurite growth, that is, the relative alignment between neurites 240 growing from the DRG 225, depends, at least in part, on the topography of the sample 200 of biomaterial underneath the neurites 240.

FIG. 13 is another image depicting the processed allograft 270 shown in FIG. 12. FIG. 13 also shows neurite growth from the DRG 225 into the allograft 270 emphasized using GAP 43 stain. In particular, the green portions of the image of FIG. 13 depict neurite growth, or neural outgrowth and regeneration, as emphasized with GAP 43 stain. As noted above, with respect to FIG. 12, the alignment of neurite growth depends, at least in part, on the topography of the sample of biomaterial underneath the neurites.

As discussed previously, bioassays described herein may take less time to conduct than traditional bioassays. For example, depending on the freezing technique used, step 105 of freezing the sample 200 of biomaterial may take minutes, and step 110 of sectioning the frozen sample 200 of biomaterial may take up to an hour or two, depending on the number of sections being generated, the width of the sections, etc. Steps 115, 120, and 125 of placing the sections 210 of biomaterial on a slide and thawing or washing the sections 210 of biomaterial may take up to a few hours, and incubation may be up to about 3 to 10 days, as described above. Staining the test construct 235 following incubation may take up to about 2 days. In all, a bioassay as described herein may take less than two weeks or less than a week to conduct, with the majority of the time being attributed with the incubation period. Further, larger numbers of sections may be generated and tested simultaneously, allowing for higher throughput of testing.

Embodiments of the description may facilitate one or more of the following: allowing for easier, faster, and higher throughput to test the potency of biomaterials and biosimilars; making neurite growth easier to quantify; allowing for sections of a sample of biomaterial to stick to slides for analysis; making it easier to see a longest neurite, or subset of longest neurites, growing in a section of the sample of biomaterial; allowing for imaging and analysis of all neurite growth in a section of the sample of biomaterial; allowing for testing and comparing effects of one or more pharmaceutical agents on neurite growth; allowing for testing of dosage amounts of one or more pharmaceutical agents; allowing for measuring of inhibitory responses of one or more pharmaceutical agents and/or devices; providing a screening tool for a bioactive; or providing a nerve model for simulating injury. Although the examples described herein include use of a biomaterial that is a nerve and use of DRGs/neurons and neurite extension, other biomaterials and growth tissue may be used.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

While principles of the present disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Furthermore, the present disclosure is not limited to the exemplary shapes, sizes, and/or materials discussed herein. Thus, a person of ordinary skill in the art will recognize that additional shapes, sizes, and/or materials may be used as discussed herein to achieve the same or similar effects or benefits as discussed above. Accordingly, the present disclosure is not to be considered as limited by the foregoing description.

We claim:

1. A method for conducting an in vitro assay of a biomaterial, the method comprising:
freezing a sample of biomaterial;
sectioning the frozen sample of biomaterial into at least one section of biomaterial, wherein the at least one section of biomaterial has a thickness of about 10 μm to about 50 μm;
placing the at least one section of biomaterial onto a slide;
affixing a neuron or a group of neurons to one end portion of the at least one section of biomaterial to create a test construct;
incubating the test construct; and
determining an amount of neurite growth from the neuron or the group of neurons along the at least one section of biomaterial.

2. The method according to claim 1, wherein determining the amount of neurite growth comprises measuring at least one longest outgrowing neurite from the neuron or the group of neurons.

3. The method according to claim 2, wherein measuring the at least one longest outgrowing neurite from the neuron or the group of neurons comprises measuring a subset of the longest outgrowing neurites and averaging the lengths of the subset of the longest outgrowing neurites.

4. The method according to claim 1, wherein the neuron or each neuron, of the group of neurons, is a dorsal root ganglia (DRG).

5. The method according to claim 1, further comprising:
staining the test construct prior to determining the amount of neurite growth from the neuron or group of neurons.

6. The method according to claim 5, wherein incubating the plurality of sections of biomaterial includes exposing the test construct to carbon dioxide.

7. The method according to claim 1, further comprising using a cryofreezing media when freezing the biomaterial.

8. The method according to claim 1, the slide has a positive charge on a surface thereof on which the at least one section is placed.

9. The method according to claim 1, further comprising thawing the at least one section of biomaterial and washing the at least one section of biomaterial with a buffer solution prior to affixing the neuron or the group of neurons.

10. The method according to claim 1, wherein the at least one section of biomaterial comprises a plurality of sections of biomaterial, and wherein each of the plurality of sections of biomaterial is placed onto a respective slide.

11. The method according to claim 10, further comprising treating a first subset of the plurality of sections of biomaterial with an agent, and further comprising determining an effect of the agent on neurite growth by comparing an amount of neurite growth in the treated first subset of sections of biomaterial to an amount of neurite growth in a second subset of untreated sections of the plurality of sections of biomaterial.

12. The method according to claim 1, wherein the biomaterial is a nerve graft.

13. A method for conducting an in vitro assay of a biomaterial, the method comprising:
freezing a sample of biomaterial using a cryofreezing media;
sectioning the frozen sample of biomaterial into a plurality of sections of biomaterial, each section having a thickness of about 10 μm to about 50 μm;
placing each section, of the plurality of sections of biomaterial, onto a slide, of a plurality of slides, wherein each slide, of the plurality of slides, has a positive charge on a surface thereof on which a section, of the plurality of sections of biomaterial, is placed;
thawing the plurality of sections of biomaterial;
washing the plurality of sections of biomaterial;
affixing a neuron or a group of neurons to one end portion of each of the plurality of sections of biomaterial;
incubating the plurality of sections of biomaterial, with the neuron or the group of neurons affixed thereto;
staining the plurality of incubated sections of biomaterial; and
determining, for each of the plurality of incubated sections of biomaterial, an amount of neurite growth from the neuron or the group of neurons along the section of biomaterial.

14. The method according to claim 13, wherein determining, for each of the plurality of incubated sections of biomaterial, the amount of neurite growth comprises measuring at least one longest outgrowing neurite from the neuron or the group of neurons.

15. The method according to claim 13, wherein the neuron or each neuron, of the group of neurons, is a dorsal root ganglia (DRG), and wherein the biomaterial is a nerve graft.

16. The method according to claim 13, further comprising freezing the plurality of sections of biomaterial on the plurality of slides prior to thawing the plurality of sections of biomaterial.

17. The method according to claim 13, wherein the plurality of slides are frosted or coated with a coating material.

18. The method according to claim 13, further comprising treating a first subset of the plurality of sections of biomaterial with an agent, and further comprising determining an effect of the agent on neurite growth by comparing an amount of neurite growth in the treated first subset of sections of biomaterial to an amount of neurite growth in a second subset of untreated sections of the plurality of sections of biomaterial.

19. The method according to claim 13, wherein incubating the plurality of sections of biomaterial occurs at a temperature of less than 40° C. for about 3 days to about 10 days.

20. A method for conducting an in vitro assay of nerve tissue, the method comprising:
freezing a sample of nerve tissue using;
sectioning the frozen sample of nerve tissue into a plurality of sections of nerve tissue, each section having a thickness of about 10 μm to about 50 μm;
placing each section, of the plurality of sections of nerve tissue, onto a positively charged slide, of a plurality of slides;
thawing the plurality of sections of nerve tissue;
washing the plurality of sections of nerve tissue;
affixing a neuron or a group of neurons to one end portion of each of the plurality of sections of nerve tissue;
incubating the plurality of sections of nerve tissue, with the neuron or the group of neurons affixed thereto, to allow for neurite growth from the neuron or the group of neurons along the nerve tissue;
staining the plurality of incubated sections of nerve tissue; and
measuring, on each slide of the plurality of slides, at least one longest outgrowing neurite from the neuron or the group of neurons along the nerve tissue.
